(19) United States
(12) Patent Application Publication
(10) Pub. No.: US 2024/0423498 A1
(43) Pub. Date: Dec. 26, 2024

(54) Estimating Tidal Volume Using Mobile Devices

(71) Applicant: Samsung Electronics Co., Ltd., Suwon-si (KR)

(72) Inventors: Md Mahbubur Rahman, San Jose, CA (US); Yincheng Jin, Port Jefferson, NY (US); Mehrab Bin Morshed, Gilroy, CA (US); Nafiul Rashid, San Jose, CA (US); Jilong Kuang, San Jose, CA (US); Jun Gao, Menlo Park, CA (US)

(21) Appl. No.: 18/752,405

(22) Filed: Jun. 24, 2024

Related U.S. Application Data
(60) Provisional application No. 63/523,272, filed on Jun. 26, 2023.
(60) Provisional application No. 63/549,707, filed on Feb. 5, 2024.

(51) Int. Cl. A61B 5/091; A61B 5/00; A61B 5/0205; A61B 5/0295; A61B 5/08; A61B 5/11

(52) U.S. Cl. CPC A61B 5/091; A61B 5/0295; A61B 5/0803; A61B 5/1102; A61B 5/6803; A61B 5/6817; A61B 5/6823; A61B 5/6898; A61B 5/7257; A61B 5/7267; A61B 5/0205; A61B 2560/0462; A61B 2562/0204; A61B 2562/0219

(57) ABSTRACT

In one embodiment, a method includes detecting, by a motion sensor of a mobile device worn by a user, multiple motion signals, each representing a motion of the user about one of a number of mobile-device axes defined by an orientation of the mobile device. The method further includes determining, for each of the multiple mobile-device axes, a ballistocardiogram (BCG) signal based on the motion signal corresponding to that mobile-device axis; selecting, based on a strength of the determined BCG signals, one or more particular mobile-device axes and corresponding motion signals for estimating a user's tidal volume; determining, based on the one or more selected motion signals, one or more breathing features; and estimating, by providing the one or more breathing features to a trained machine-learning model, the user's current tidal volume.

### PRIORITY CLAIM

This application claims the benefit under 35 U.S.C. § 119 of U.S. Provisional Patent Application No. 63/523,272 filed Jun. 26, 2023, and of U.S. Provisional Patent Application No. 63/549,707 filed Feb. 5, 2024, each of which are incorporated by reference herein.

### TECHNICAL FIELD

This application generally relates to estimating tidal volume using mobile devices.

### BACKGROUND

Tidal volume (Vt) is the volume of air that a person inhales while breathing. Tidal volume is one of the main determinants of minute ventilation and alveolar ventilation. Minute ventilation, also known as total ventilation, is a measurement of the amount of air that enters the lungs per minute, e.g., minute ventilation equals respiratory rate times tidal volume. Alveolar ventilation, also known as actual ventilation, represents the volume of air that reaches the respiratory zone per minute, e.g., alveolar ventilation equals respiratory rate times (tidal volume-dead space).

Accurately determining a person's tidal volume can help detect hypoventilation or hyperventilation symptoms. Hypoventilation occurs when a person is breathing too slow or too shallow, and can result from, e.g., drowsiness, drug overdose, fatigue, illness, and so on. Hyperventilation occurs when a person is breathing too fast or too deep, and can result from, e.g., anxiety, dizziness, weakness, and so on.

A person's tidal volume is related to many health conditions, some of them critically important. For example, the respiration rate divided by tidal volume, which is known as the rapid shallow breathing index, is an important biomarker for predicting congestive heart failure. As another example, a person with a restrictive lung disease needs to use rapid, shallow breathing with lower tidal volume. As another example, a person with an obstructive lung disease (e.g., COPD, asthma), needs to use slow, deep breathing with higher tidal volume. As another example, when mechanical ventilation is used, tidal volume should be kept within a particular range to avoid lung injury. Finally, a person with a neuromuscular disease typically shows symptoms of rapid, shallow breathing with lower tidal volume.

### BRIEF DESCRIPTION OF THE DRAWINGS

FIG. 1 illustrates a motion sensor's coordinates system in Cartesian coordinates relative to a user's superior-inferior axis.

FIG. 2 illustrates an example method for selecting one or more motion signal(s) from one or more corresponding motion-sensor axes that best represents that user's breathing motion.

FIG. 3 illustrates an example implementation of the example method of FIG. 2

FIG. 4 illustrates an example output of static segmentation block 310.

FIG. 5 illustrates an example IJK signal.

FIG. 6 illustrates an example accelerometer output taken when a user is sitting and an example accelerometer output taken when a user is lying down.

FIG. 7 illustrates an example embodiment that uses recorded audio to estimate tidal volume.

FIG. 8 illustrates an example embodiment in which both motion data and audio are used to estimate tidal volume.

FIG. 9 illustrates an example embodiment in which both motion data and audio can be collected from a mobile device, such as a phone or a watch held to a user chest for a period of time.

FIG. 10 illustrates an example embodiment that uses a captured PPG signal to estimate tidal volume.

FIG. 11 illustrates an example computing system.

FIG. 12 illustrates an example for selecting an axis that best represents a user's breathing motion.

### DESCRIPTION OF EXAMPLE EMBODIMENTS

Conventional tidal volume (Vt) monitoring approaches include spirometry or capnography. These approaches require a person to wear an intrusive and uncomfortable mask or mouthpiece that can prompt undesirable alterations in respiratory patterns. In addition, chest straps worn round the chest and abdomen can be used to estimate tidal volume, but such straps are uncomfortable and expensive, and are not very accurate. Moreover, all of these techniques require some burdensome activity (e.g., wearing a mask, putting on a chest strap) that disrupts a person's daily activities. In practice, these deficiencies in conventional Vt monitoring have resulted in such monitoring rarely being performed due to the lack of accurate, but also unobtrusive, monitoring technologies.

In contrast, this disclosure describes techniques for noninvasive and continuous real-time Vt monitoring. Breathing conditions (e.g., respiratory rate, etc.) can be determined by analyzing motion signals from a motion sensor of a mobile device worn by a user. For example, U.S. patent application Ser. No. 18/198,989 describes several techniques for estimating breathing conditions from motion signals from a motion sensor of a mobile device, such as from a pair of earbuds worn by the user. However, in general a user's breathing-related motions are concentrated along particular axes of the body, e.g., the motion due to breathing in and out is strongest in the axis that is aligned through the length of the body, from the user's head down to the feet (i.e., along the superior-inferior axis). Therefore, motion along this axis is the most useful and predictive from which to determine breathing features; if motion from a perpendicular axis (e.g., one that runs the through user's head, from ear to ear) is instead considered, then the breathing conditions will be less accurately estimated, as motion along this axis contains relatively little information about the user's breathing.

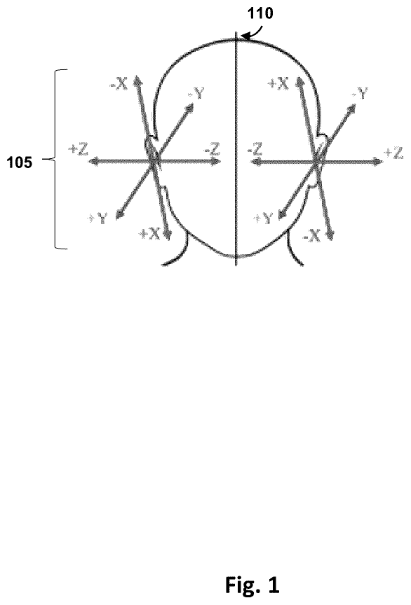

However, the orientation of a worn mobile device's orientation is not known in real-time relative to the user's body, nor are these relative orientations static. For example, FIG. 1 illustrates a motion sensor's coordinates system 105 in Cartesian coordinates relative to a user's superior-inferior axis 110. The sensor's coordinate system remains the same in its own frame of reference, but the relative orientation between coordinate system 105 and the coordinate system of the user's body (e.g., the relative orientation of coordinate system 105 and of superior-inferior axis 110) varies based on exactly how the user is wearing the earbuds, e.g., at what orientation the user placed the earbuds into their cars. For example, rotating the earbuds in the ears will change the orientation of coordinate system 105 relative to the orientation of the user's body, such that an axis that may have been well aligned with superior-inferior axis 110 in one earbud position is no longer well aligned in another. In addition, a user may vary their head angle relative to the body (e.g., tilt their head back, forward, or side-to-side) and while such activities do not change the superior-inferior axis 110, they do change the orientation of the motion sensor's coordinate system 105, and therefore an axis in coordinate system 105 that aligns well with superior-inferior axis 110 when a user is looking straight ahead may not align well when the user is tilting their head. Selecting motion signals from one or more predetermined axis from which to derive breathing conditions will result in inaccuracies due to variances in how the user orients the wearable device containing the motion sensor, and in how the user orients the portion of the body in which the wearable device is worn relative to the rest of the body.

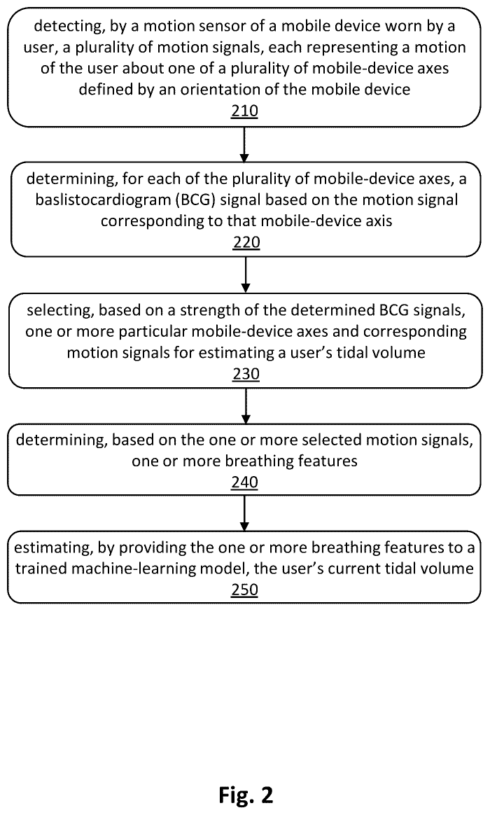

Certain techniques of this disclosure dynamically identify one or more suitable motion-sensor axes, in real-time, from which corresponding motion data can accurately determine a user's breathing conditions, such as tidal volume. FIG. 2 illustrates an example method for selecting one or more motion signal(s) from one or more corresponding motion-sensor axes that best represents that user's breathing motion, i.e., the motion about such axes that contains the most motion information from the user's breathing.

Step 210 of the example method of FIG. 2 includes detecting, by a motion sensor of a mobile device worn by a user, a plurality of motion signals, each representing a motion of the user about one of a plurality of mobile-device axes defined by an orientation of the mobile device. For example, the motion sensor may be an accelerometer, and the mobile device may be a head-worn device (e.g., an earbud, earphones, headset (e.g., XR headset), glasses, etc.). As described more fully below, other embodiments may use mobile devices that are worn other than on the head, e.g., may use a mobile phone or a watch that is held against the user' body (e.g., held against the user's chest). FIG. 1 illustrates an example of a plurality of mobile-device axes (e.g., the Cartesian x,y,z axes) defined by an orientation of the mobile device, although other coordinate representations may be used.

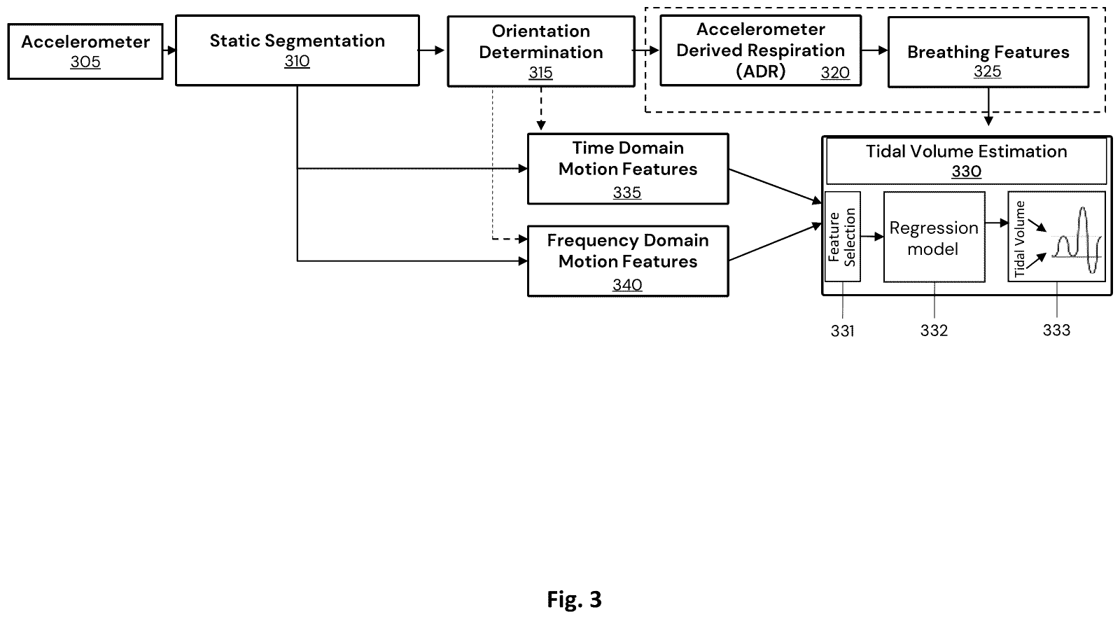

The motion sensor, such as the accelerometer, collects motion data in its frame of reference. FIG. 3 illustrates an example implementation of the example method of FIG. 2. In the example of FIG. 3, accelerometer data from accelerometer 305 is passed to static segmentation block 310, which outputs a segment of motion data to orientation-determination block 315. As described below, in particular embodiments the motion signals of step 210 may be the motion signals corresponding to the output of static segmentation block 310, i.e., may be the motion signals in the static motion segment as determined by block 310.

Attempting to analyze breathing data from motion signals that also include other movement of the user (e.g., when the user is running, dancing, walking, etc.) may result in inaccurate breathing-condition estimates, as the user's other motion may dwarf the motion due to breathing. Static segmentation block 310 addresses this problem by selecting a static motion segment from accelerometer 305's output as follows. First, the accelerometer output is divided into intervals of a predetermined length, e.g., 0.5 seconds each. Then, static segmentation block 310 uses a threshold to categorize each half-second interval as either motion or static. For instance, the threshold may be based on energy expenditure (EE) in the motion signal interval, where:

$$E E = \frac{\sqrt{{( {x}_{i + 1} - {x}_{i} )}^{2} + {( {y}_{i + 1} - {y}_{i} )}^{2} + {( {z}_{i + 1} - {z}_{i} )}^{2}}}{number of samples}$$

where i denotes the position of the data being calculated. If the EE calculated for an interval exceeds the threshold (which may be determined, for example, by calculating the average value of data within the inter-quartile range), then that interval is identified as dynamic motion (which may, for example, correspond to an assigned value of 1). Conversely, if the current EE for an interval is less than or equal to the threshold, then this interval is identified as static (which may, for example, correspond to an assigned value of 0). In this example, static segmentation block 310 produces a sequence of labelled intervals in the motion signal output by accelerometer 305. Here, the output of accelerometer 305 may refer to the raw output, or a processed version of the raw signal.

Static segmentation block 310 may then identify the longest sequence of continuous intervals identified as a static signal (e.g., the longest continuous sequence of “0” labels). If the duration of this sequence exceeds a predetermined threshold time, then the motion-signal output of accelerometer 305 corresponding to this sequence is selected for subsequent feature extraction. In particular embodiments, the predetermined threshold time may be, e.g., around 10 seconds, which corresponds to at least three breathing cycles. As described in this example, a predetermined threshold time may be based on a number of breathing cycles to include in the motion signal, as more breathing cycles will typically result in better feature extraction; on the other hand, more breathing cycles results in a longer sequence, and therefore a more demanding requirement that such sequence be static.

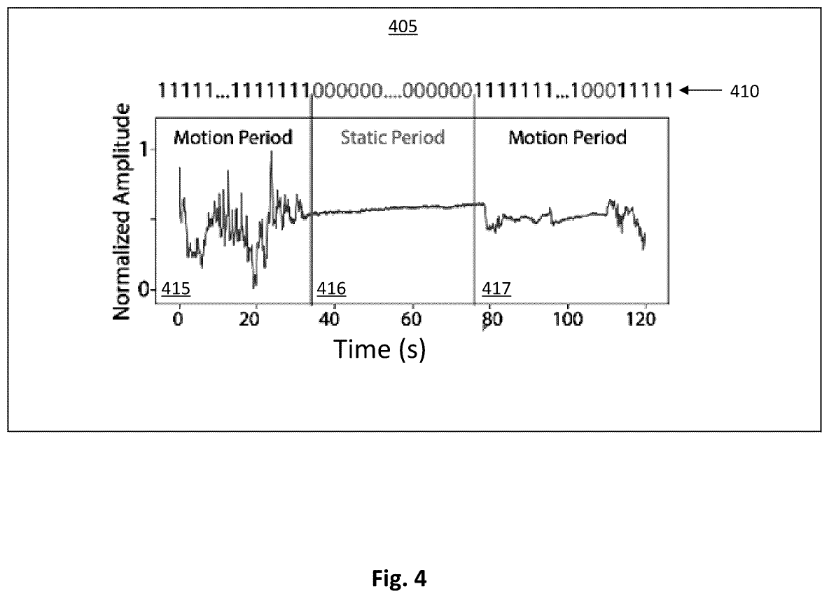

FIG. 4 illustrates an example output of static segmentation block 310. Graph 405 illustrates a sequence of labels 410, each corresponding to an interval of motion data. Motion segments 415, 416, and 417 are labelled based on the labels 410 of the intervals each segment contains. As illustrated in the example of FIG. 4, segment 416 is identified as a static segment due to the continuous sequence of static labels that exceed the predetermined threshold time. In addition, as illustrated in FIG. 4, segment 417 contains both static and dynamic motion labels, but is nevertheless identified as a motion (non-static) segment due to the fact that the continuous static intervals in segment 417 are not sufficiently long enough to meet the predetermined threshold time.

After a suitable static segment is identified, then various de-noising or processing steps may be applied to the motion signals in that segment. For example, such processing may include applying a median filter to smooth the signals, and then applying a standard normalization to each sensor channel (e.g., the motion signal of each of the 3 x, y, and z axes) to achieve consistent values. Filtering the signals helps reduce noise, among other things, thereby facilitating extraction of breathing features.

Step 220 of the example method of FIG. 2 includes determining, for each of the plurality of mobile-device axes, a ballistocardiogram (BCG) signal based on the motion signal corresponding to that mobile-device axis. The BCG signal is most prominent along the superior-inferior axis of a person's body. In general, the strongest BCG responses are observed across the superior inferior axis, followed by the anterior posterior axis, and then lastly the lateral medial axis.

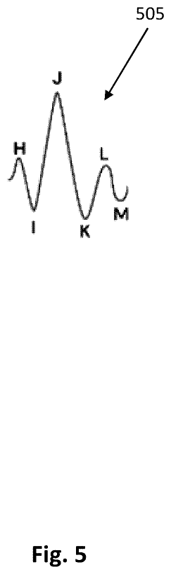

The heart pumping blood through arteries creates body motion, which is called the BCG response, and also creates corresponding BCG morphological signal complex called IJK. FIG. 5 illustrates an example IJK signal 505. As illustrated in FIG. 5, various peaks and valleys are labeled from H to M, with the highest peak in the IJK signal identified as the J peak.

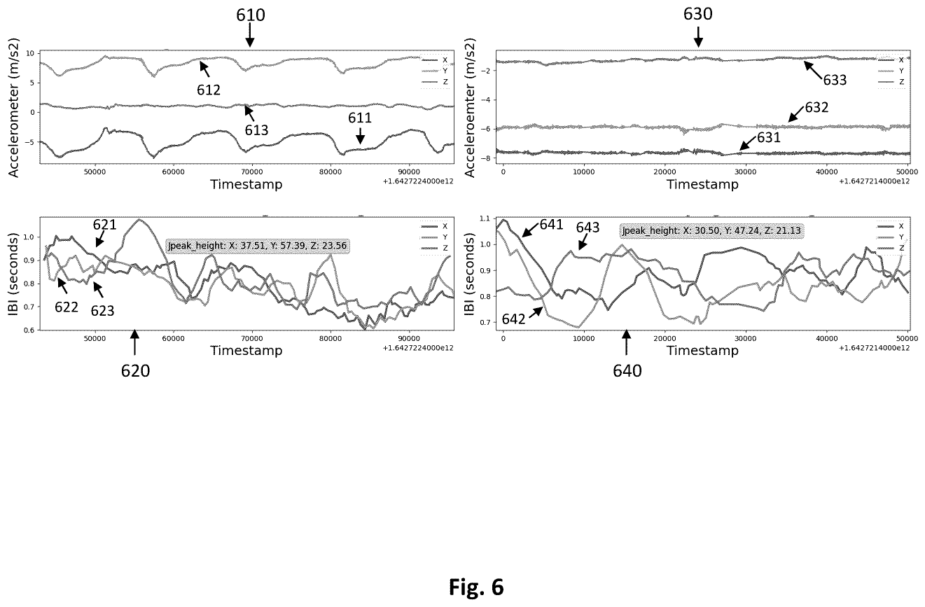

Step 220 may include determining the J-peak height for a BCG signal corresponding to each accelerometer axis. FIG. 6 illustrates an example accelerometer output 610 taken when a user is sitting and an example accelerometer output 630 taken when a user is lying down. Accelerometer output 610 includes motion signal 611 corresponding to the accelerometer's x axis, motion signal 612 corresponding to the accelerometer's y axis, and motion signal 613 corresponding to the accelerometer's z axis. Likewise, accelerometer output 630 includes motion signal 631 corresponding to the accelerometer's x axis, motion signal 632 corresponding to the accelerometer's y axis, and motion signal 633 corresponding to the accelerometer's z axis.

Each motion signal (e.g., each of signals 611, 612, and 613) in the motion sensor's output is analyzed to determine the J-peaks corresponding to the BCG signal. Any suitable approach for detecting J peaks from the motion signal may be used, such as for example the techniques described in D. J. Lin et al., “Ballistocardiogram-Based Heart Rate Variability Estimation for Stress Monitoring using Consumer Earbuds,” ICASSP 2024-2024 IEEE International Conference on Acoustics, Speech and Signal Processing (ICASSP), Seoul, Korea, Republic of, 2024, pp. 2111-2115, which description is incorporated by reference herein. For instance, the inter-beat-interval (IBI) can be estimated based on the motion signal. Processing may be performed on the IBI estimates, for example by removing IBIs affected by motion artifacts and correcting some of the erroneous IBI by estimating instantaneous heart rate and considering a Gaussian prior, and then each J-peak can be identified as the highest peak in the vicinity of each IBI estimate. J-peaks can be detected in the motion signal with very high (e.g., 95% or greater) accuracy.

Graph 620 illustrates an IBI estimate made for each motion signal illustrated in output 610. Specifically, BCG IBI estimate 621 corresponds to motion signal 611, BCG IBI estimate 622 corresponds to motion signal 612, and BCG IBI estimate 623 corresponds to motion signal 613. Likewise, graph 640 illustrates an IBI estimate made for each motion signal illustrated in output 630. Specifically, BCG IBI estimate 641 corresponds to motion signal 631, BCG IBI estimate 642 corresponds to motion signal 632, and BCG IBI estimate 643 corresponds to motion signal 633. In other words, for each of the x, y, z, axes in the example accelerometer's frame of reference, motion signals are captured, and each of these motions signals are used to estimate a corresponding BCG IBI signal.

Step 230 of the example method of FIG. 2, includes selecting, based on a strength of the determined BCG signals, one or more particular mobile-device axes and corresponding motion signals for estimating a user's tidal volume. For instance, with reference to the example of 610 and 620 in FIG. 6, the J-peak magnitude (strength) for each signal (e.g., for signal 621, 622, and 623 in the example of 620) are separately determined. Because the strength of the J-peak corresponds to the strength of the BCG signal, and the strength of the BCG signal is axis dependent in the same way that motion due to breathing is axis dependent (i.e., both phenomena show the strongest signal in the superior-inferior axis), the motion signals can be ordered by their J-peak strength. In particular embodiments, the motion signal and corresponding axis with the strongest J-peak signal can be selected as the motion signal in step 230. For instance, if signal 621 corresponding to the BCG IBI signal from the accelerometer's x axis contains the strongest J peak, then motion data about the x axis (e.g., motion data 611 or 621, as explained below) can be selected for further breathing analysis, based on the inference that the x-axis in fact best aligns with the superior-inferior axis of the user. On the other hand, if signal 622 corresponding to motion about the accelerometer's y-axis shows the strongest J-peak signal, then motion data about the y axis (e.g., motion data 612 or 622) can be selected for further breathing analysis, based on the inference that the y-axis in fact best aligns with the superior-inferior axis of the user.

Particular embodiments may determine a projection matrix based on the J-peak magnitude from the BCG IBI signals determined for each motion-sensor axis. The projection matrix transforms the accelerometer's x, y, z axis to the x axis (superior inferior axis), y axis (anterior posterior axis), and z axis (lateral medial axis) of the body. For example, if the strongest BCG IBI signal is determined to be about the z, x, and y axes in the accelerometer's frame of reference, in that order, then a projection matrix may be:

$${[ \begin{matrix}X \\ Y \\ Z\end{matrix} ]}_{body} = [ \begin{matrix}0 & 0 & 1 \\ 1 & 0 & 0 \\ 0 & 1 & 0\end{matrix} ] \times {[ \begin{matrix}X \\ Y \\ Z\end{matrix} ]}_{accelerometer}$$

Particular embodiments may select more than one axis (and therefore more than one motion signal) to use for estimating a user's tidal volume. For example, particular embodiments may select the top 2 axes and corresponding motion signals from which to estimate breathing features. Particular embodiments may apply a weighted average to this selection (e.g., the affect of each motion signal may be weighted by that motion signal's relative J-peak strength). Particular embodiments may intentionally not use all three axes, as the motion information about the lateral medial axis in particular may not be meaningfully related to breathing motion. In addition to resulting in more accurate determinations of breathing features (and therefore of tidal volume estimate), the techniques described herein may require less computational resources, by removing the need to process motion signals about one or more axes output by the motion sensor.

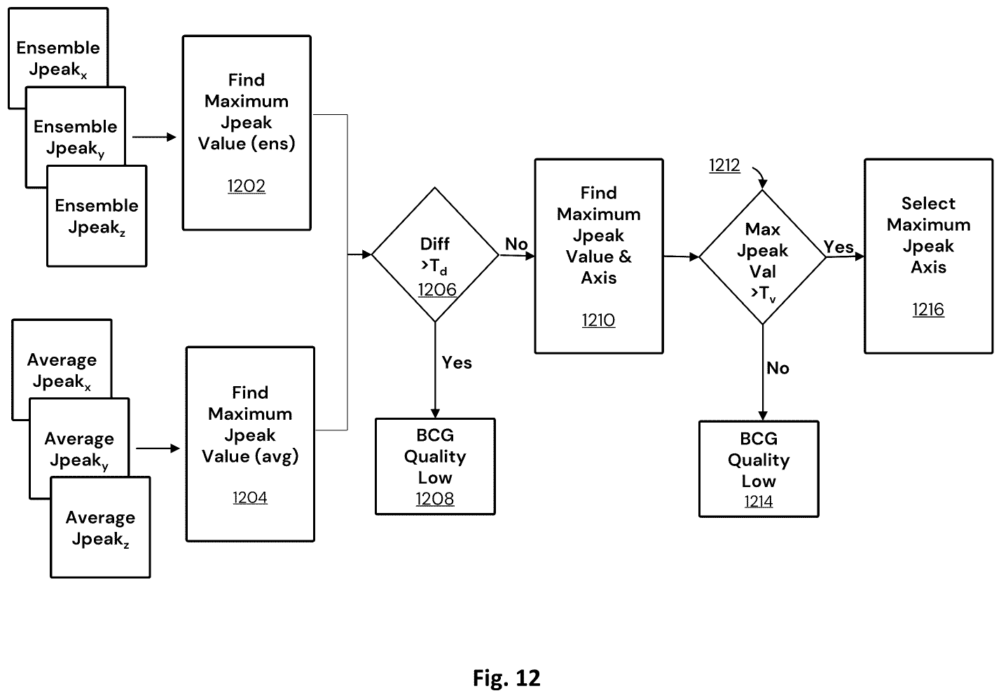

Particular embodiment can calculate J-peak magnitude (strength) for each three axis (X, Y, Z) by one or more of the following two methods—(1) by averaging all the individual J-peaks detected in a predefined time period (e.g., a minute), or (2) through an ensemble approach by overlaying each BCG beat centered at J-peak position. Particular embodiments can compare the J-peak magnitude from those two methods whether they are close to each other, i.e., the difference is lower than a threshold (e.g., 3.0). If yes, particular embodiments can find the axis that has maximum J-peak magnitude. If this J-peak magnitude value is above a certain threshold (e.g., 10.0), then particular embodiments can select this axis as the best axis among all. If the current time window does not satisfy the quality thresholds, then particular embodiments can discard the window and continue searching for the next static window that satisfies the quality thresholds. FIG. 12 illustrates a corresponding example for selecting an axis that best represents a user's breathing motion. First, the average Jpeak and the ensemble Jpeak are determined for each axis from the given set of data. Step 1202 includes determining the maximum Jpeak value from the ensemble approach, and step 1204 includes determining the maximum Jpeak value from the average approach. Step 1206 compares the differences between the two maximum Jpeak values determined in steps 1202 and 1204. If the difference is less than a threshold T_{d} (e.g., 3.0), then step 1210 includes finding the axis that has the maximum Jpeak value; otherwise, the BCG quality in the data sample is determined to be too low in step 1208. The decision block in step 1212 includes determining whether the maximum Jpeak value for the selected axis is above a particular threshold Tᵥ; if yes, then the Jpeak axis determined in step 1210 is the selected axis, i.e., the axis along which the BCG signal is the strongest. Otherwise, the BCG quality is determined to be too low to use in step 1214.

Posture can have a significant impact on a motion sensor's response due to breathing motion. For example, when a person lying down, the breathing motion intensity captured by an earbud accelerometer will be lower than when the person is sitting upright position. Examples 610 (motion while sitting) and 630 (motion while lying) illustrate these differing motion intensities. While lying, a person's head motion will be limited due to damping of the motion caused by contact between the head and another object, and in these instances, using the BCG motion signal (e.g., signal 640) may be more accurate than using the accelerometer motion signal (e.g., 630) to determine breathing features. However, while sitting or standing, the motion signal from the accelerometer may be stronger, and therefore using this signal rather than the corresponding BCG signal may lead to more accurate subsequent breathing-feature extraction. Particular embodiments can determine which motion signal to use for subsequent feature extraction based on the user's posture, which posture may be determined by, for example, the techniques described in U.S. patent application Ser. No. 18/198,989, which is incorporated herein by reference. Particular embodiments may calibrate the accelerometer signal based on the posture to amplify the accelerometer features for more accurate estimation.

People naturally take deeper breaths after intense physical activities. Therefore, the tidal volume can be higher during and after activities compared to the resting state. In those cases, breathing motions become stronger than when at rest. Particular embodiments may detect physical activities such as walking, jogging, running, cycling (e.g., using the motion sensor of the mobile device or using other techniques) and determine the tidal volume accordingly. Particular embodiments may tag the tidal volume with associated activities and categorize the numbers based on their activities for better insights into changes in users' tidal volumes.

Step 240 of the example method of FIG. 2 includes determining, based on the one or more selected motion signals, one or more breathing features of the user. For instance, the example implementation of FIG. 3 includes Accelerometer Derived Respiration (ADR) block 320, which determines breathing features 325 from the selected accelerometer motion signal(s) that were selected in step 230. Breathing features may include breathing amplitude, breathing frequency, the ratio between the inhalation and exhalation phase (symmetry), etc. U.S. patent application Ser. No. 18/198,989 describes several techniques for extracting breathing features from accelerometer motion signals, and this description is incorporated by reference herein. Other embodiments may use the respiration signal (e.g., as illustrated in graphs 620 and 640) from which to determine one or more breathing features of the user. For instance, as described above, the BCG respiration signal may be used when the user is lying down. The extracted breathing features are the same as those described above with respect to the ADR signal.

To extract breathing features, particular embodiments use a peak-detection algorithm to find the peaks and valleys in the filtered signal (whether ADR signal or BCG signal). Each valley-to-peak indicates an inhale cycle, and the peak-to-valley indicates an exhale cycle. In an ideal breathing cycle, peaks and valleys must occur in an alternating order. However, false peaks or valleys can be detected due to small noises in the derived breathing cycle. For that reason, false peaks and valleys are removed if there are multiple peaks in between two valleys or multiple valleys in between two peaks. When there are multiple peaks in between two valleys, particular embodiments select the ones that are closest to the nearest peak.

Once the correct peaks and valleys are identified, particular embodiments can extract breathing features including breathing depth (amplitude), rate, and symmetry. Breathing depth shows one of the strongest correlations with tidal volume; however, breathing rate and symmetry can also be important features for tidal volume estimation.

Step 250 of the example method of FIG. 2 includes estimating, by providing the one or more breathing features to a trained machine-learning model, the user's current tidal volume. FIG. 3 illustrates an example implementation in which breathing features 325 are provided to a tidal volume estimation model 330. As illustrated in the example of FIG. 3, particular embodiments may also estimate additional motion features, such as time-domain motion features 335 and frequency domain motion features 340, from the static motion signal output by static segmentation block 310, and these motion features are input to tidal volume estimation model 330, which performs features selection 331 on these input motion features. Time-based features may include e.g., mean and standard error of the input motion signal, and frequency-based features may include, e.g., spectral power and entropy of the input motion signal. Particular embodiments may train a feature-selection model to determine which features to select from time-based or frequency-based motion features.

In particular embodiments, either or both of time-domain and frequency-domain motion features may be determined from all of the motion sensor's static output, e.g., from motion about all three axes of an accelerometer. This approach may result in a richer set of features selected by feature selection 331, and therefore more accurate estimates of tidal volume. However, particular embodiments may use orientation determination model 315 to determine which motion signals should be used to determine time-domain and frequency-domain motion features. For instance, such features may be determined based on motion signals about only one axis, or fewer than all three axes, thereby reducing computational resources needed to determine and select such features.

After obtaining the relevant set of the extracted features, particular embodiments may apply features selection, for example by applying a Gini-importance-based feature selection to reduce the feature-vector size to the most important features, e.g., 20 features. These features are then fed into a trained machine learning model, such as a regression model, to estimate the tidal volume.

FIG. 3 illustrates an example machine-learning regression model 332 for performing tidal volume estimates 333 based on input features, which include breathing features 325 and may include one or more of time-domain features 335 and frequency-domain features 340. An example, regression model may include three fully connected layers that receive input data (i.e., 20 features, such as mean, max, min, breath features) with the shape specified by the input shape. The first layer is the input layer, which may consist of 256 units with a Rectified Linear Unit (ReLU) activation function and uniform weight initialization. To help prevent overfitting of this model, a dropout layer with a dropout rate of 0.25 may operate in conjunction with the input layer. The dropout rate of 0.25 means that this layer randomly sets 25% of the input values to 0. The second layer may include 64 units with a ReLU function and a dropout layer with a dropout rate of 0.25. A batch normalization function may be included to normalize the activations of the previous layer at each batch, thereby reducing internal covariate shift. Since this example model is a regression model, a single dense layer is adopted with a linear activation function. This example model may utilize a mean squared error as the loss function and the Adam optimizer for training with a learning rate of, e.g., 0.001.

Particular embodiments may repeat one or more steps of the method of FIG. 2, where appropriate. Although this disclosure describes and illustrates particular steps of the method of FIG. 2 as occurring in a particular order, this disclosure contemplates any suitable steps of the method of FIG. 2 occurring in any suitable order. Moreover, although this disclosure describes and illustrates particular components, devices, or systems carrying out particular steps of the method of FIG. 2, this disclosure contemplates any suitable combination of any suitable components, devices, or systems carrying out any suitable steps of the method of FIG. 2. Moreover, this disclosure contemplates that some or all of the computing operations described herein, including certain steps of the example method illustrated in FIG. 2, may be performed by circuitry of a computing device described herein, by a processor coupled to non-transitory computer readable storage media, or any suitable combination thereof.

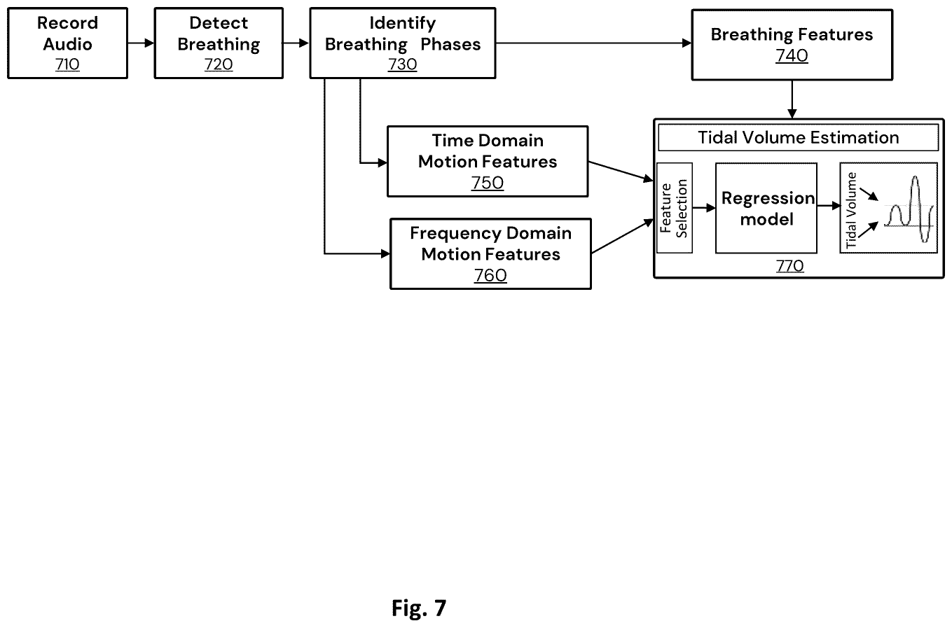

FIG. 7 illustrates an example embodiment that uses recorded audio to estimate tidal volume. Breathing audio is a relatively lower intensity sound compared to other environmental noise (e.g., speech), but some devices (e.g., an earbud, a headset, etc.) are close to the mouth and nose and therefore better capture the breathing sound than other devices that are farther away from the body. Moreover, a device may be held up to the nose and mouth, thereby capturing audio due to breathing. Block 710 includes recording audio by an acoustic sensor, and block 720 includes detecting audio that corresponds to breathing. Step 730 includes identifying breathing phases (e.g., inhale-exhale phases), for example as described in U.S. patent application Ser. No. 18/358,769, which description is incorporated by reference herein. Step 740 includes detecting breathing features (e.g., phase duration, rate, phase amplitude) from the identified breathing phases, and these features are fed (along with, optionally, one or more of time-domain motion features 750 (e.g., Zero Crossing Rate (ZCR), Area Under Envelope (AUE)) and frequency-domain motion features 760 (e.g., Mel Frequency Cepstral Coefficients (MFCC), Chroma, entropy, energy, spectral density)) to tidal volume estimation model 770, which may be the architecture described above with reference to tidal volume estimation model 330.

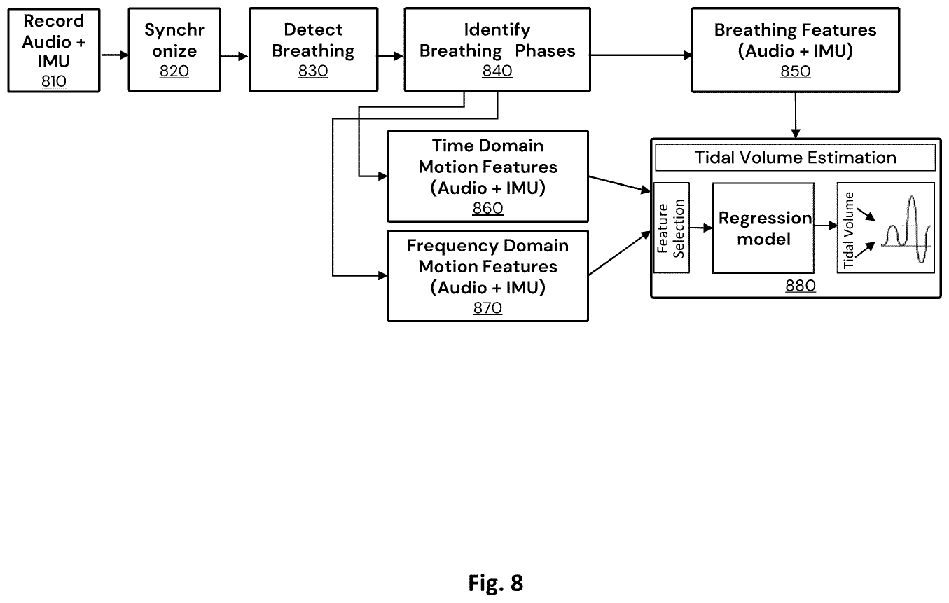

FIG. 8 illustrates an example embodiment in which both motion data and audio are captured, e.g., from an earbud, which are then synchronized to identify the breathing phases. Block 810 includes recording audio and motion data, and block 820 includes synchronizing this collected data. Synchronization can be done based on identified breathing phases, as inhalation and exhalation breathing motion can be obtained from accelerometer derived respiration (ADR) after performing orientation detection. After detecting breathing in block 830 and identifying breathing phases in block 840, breathing features can then be extracted in block 850, where the features are derived from the audio and IMU data. These features, optionally along with one or more of time-domain features 860 and frequency-domain features 870, may be provided to tidal volume estimation model 880. Particular embodiments may select between the audio and IMU features based on the current context to maximize the accuracy and the yield for tidal volume estimation.

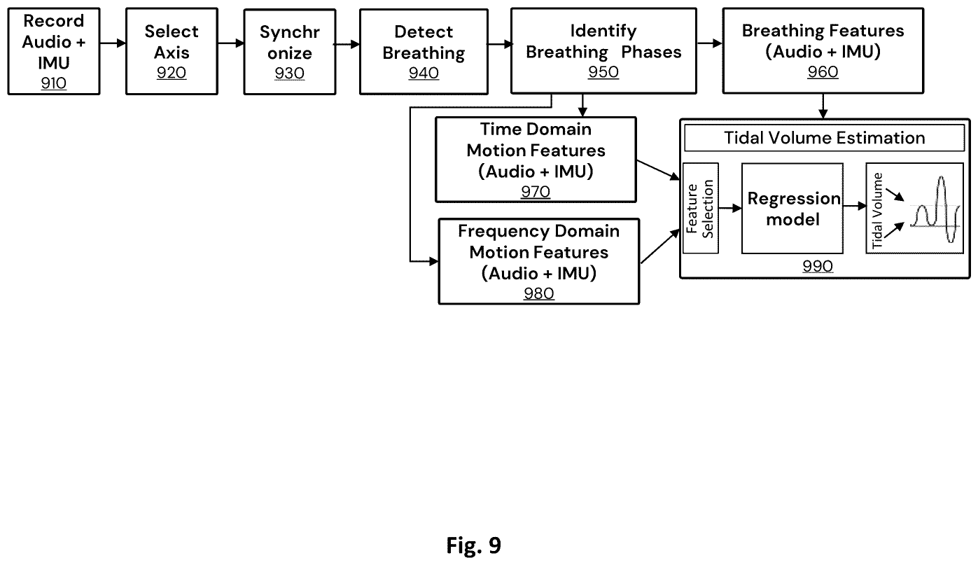

FIG. 9 illustrates an example embodiment in which both motion data and audio can be collected from a mobile device, such as a phone or a watch or a smart ring held to a user chest for a period of time, e.g., one minute. Both audio and motion (IMU) data are recorded in block 910, and axis selection based on orientation detection occurs in block 920, followed by synchronization in block 930. Breathing and breathing phases are detected in blocks 940 and 950, respectively, from which breathing features can be extracted in block 960. These breathing features, optionally along with one or more of time-domain features 970 and frequency-domain features 980, may be provided to tidal volume estimation model 990.

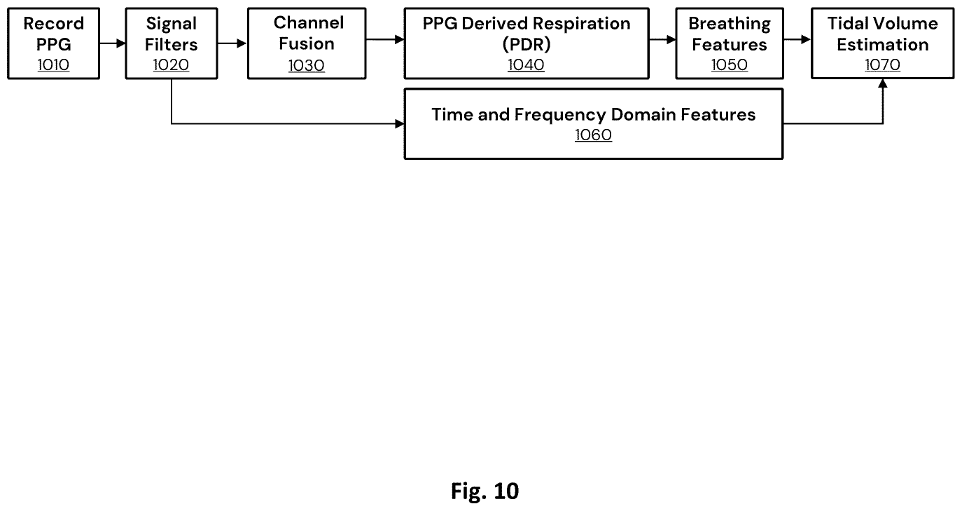

FIG. 10 illustrates an example embodiment that involves passively capturing a photoplethysmograph (PPG) signal from a device such as a watch or earbuds to determine a respiratory signal from the baseline wander, amplitude modulation, and frequency modulation once the PPG signal is above a certain quality threshold and motion artifacts are minimal. A PPG signal captures respiration since breathing affects heart beat. Heart beat increases during inhalation and decreases during exhalation. This phenomenon is called respiratory sinus arrythmia. Particular embodiments can extract this signal by detecting the PPG peaks and extracting the trend of the inter-beat-interval from the PPG peaks. Moreover, breathing causes baseline wander in the PPG signal and also affects the amplitude of the PPG peaks. Particular embodiments combine all these derived respiratory signals and extract breathing features from each of them to train a regression model for tidal volume estimation. For example, block 1010 includes recording a PPG signal, filtering the signal in block 1020, and performing PPG channel fusion in block 1030 to determine a PPG derived respiration (PDR) in block 1040. The PDR is analyzed to extract breathing features in block 1050, and these features (optionally along with one or more of time-domain features and frequency-domain features 1060, each derived from the filtered PPG signal), may be provided to tidal volume estimation model 1070 to estimate a user's tidal volume.

Wearable devices may be used to estimate tidal volume and therefore diagnose, detect, and monitor a number of related health conditions of a user. For example, estimates of tidal volume may be used to detect all three types of sleep apnea based on breathing depth threshold where breathing depth during obstructive sleep apnea and central sleep apnea will be close to zero, and breathing depth will be between zero and normal breathing for hypopnea. Tidal volume estimates may be used in critically ill patients on a ventilator, in order to deliver a tidal volume large enough to maintain adequate ventilation but small enough to prevent lung trauma. Tidal volume estimates may be used to detect breathlessness or shortness of breath, which are associated with several medical emergencies. As another example, tidal volume over respiratory rate (Vt/RR) is a key indicator sign associated with cardiac arrest, and is an exacerbating condition for heart failure. Tidal volume estimates may be used to detect apnea in patients, which may occur due to various medical conditions or ingestion of toxins (e.g., carbon monoxide). In addition, tidal volume monitoring may be beneficial to identify abnormal breathing patterns that warrant medical emergencies, such as agonal breathing, choking, asthma/COPD exacerbation, etc. Other example benefits of tidal volume monitoring include hyperventilation and hypoventilation assessment, VO2max estimation, and respiratory fitness management for healthy individuals (e.g., to improve a user's breathing techniques, during an exercise activity or in general).

The techniques described herein may also be useful for more accurately identifying breathing features detected by a worn mobile device, and this improved accuracy can be beneficial in detecting and monitoring a wide variety of medical conditions.

If a condition is detected, then particular embodiments may send an alert or other notification to the user (e.g., by a sound or a displayed UI), to a medical or emergency professional, and/or to a medical contact, etc. Particular embodiments may also or in the alternative present information (e.g., audibly or visually) to a user regarding the medical emergency.

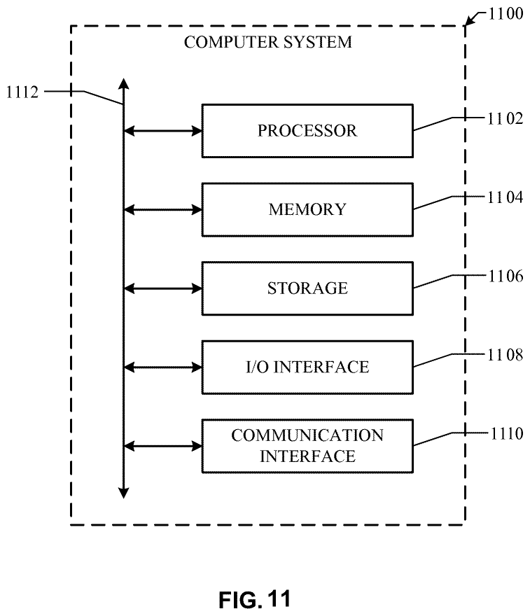

FIG. 11 illustrates an example computer system 1100. In particular embodiments, one or more computer systems 1100 perform one or more steps of one or more methods described or illustrated herein. In particular embodiments, one or more computer systems 1100 provide functionality described or illustrated herein. In particular embodiments, software running on one or more computer systems 1100 performs one or more steps of one or more methods described or illustrated herein or provides functionality described or illustrated herein. Particular embodiments include one or more portions of one or more computer systems 1100. Herein, reference to a computer system may encompass a computing device, and vice versa, where appropriate. Moreover, reference to a computer system may encompass one or more computer systems, where appropriate.

This disclosure contemplates any suitable number of computer systems 1100. This disclosure contemplates computer system 1100 taking any suitable physical form. As example and not by way of limitation, computer system 1100 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, or a combination of two or more of these. Where appropriate, computer system 1100 may include one or more computer systems 1100; be unitary or distributed; span multiple locations; span multiple machines; span multiple data centers; or reside in a cloud, which may include one or more cloud components in one or more networks. Where appropriate, one or more computer systems 1100 may perform without substantial spatial or temporal limitation one or more steps of one or more methods described or illustrated herein. As an example and not by way of limitation, one or more computer systems 1100 may perform in real time or in batch mode one or more steps of one or more methods described or illustrated herein. One or more computer systems 1100 may perform at different times or at different locations one or more steps of one or more methods described or illustrated herein, where appropriate.

In particular embodiments, computer system 1100 includes a processor 1102, memory 1104, storage 1106, an input/output (I/O) interface 1108, a communication interface 1110, and a bus 1112. Although this disclosure describes and illustrates a particular computer system having a particular number of particular components in a particular arrangement, this disclosure contemplates any suitable computer system having any suitable number of any suitable components in any suitable arrangement.

In particular embodiments, processor 1102 includes hardware for executing instructions, such as those making up a computer program. As an example and not by way of limitation, to execute instructions, processor 1102 may retrieve (or fetch) the instructions from an internal register, an internal cache, memory 1104, or storage 1106; decode and execute them; and then write one or more results to an internal register, an internal cache, memory 1104, or storage 1106. In particular embodiments, processor 1102 may include one or more internal caches for data, instructions, or addresses. This disclosure contemplates processor 1102 including any suitable number of any suitable internal caches, where appropriate. As an example and not by way of limitation, processor 1102 may include one or more instruction caches, one or more data caches, and one or more translation lookaside buffers (TLBs). Instructions in the instruction caches may be copies of instructions in memory 1104 or storage 1106, and the instruction caches may speed up retrieval of those instructions by processor 1102. Data in the data caches may be copies of data in memory 1104 or storage 1106 for instructions executing at processor 1102 to operate on; the results of previous instructions executed at processor 1102 for access by subsequent instructions executing at processor 1102 or for writing to memory 1104 or storage 1106; or other suitable data. The data caches may speed up read or write operations by processor 1102. The TLBs may speed up virtual-address translation for processor 1102. In particular embodiments, processor 1102 may include one or more internal registers for data, instructions, or addresses. This disclosure contemplates processor 1102 including any suitable number of any suitable internal registers, where appropriate. Where appropriate, processor 1102 may include one or more arithmetic logic units (ALUs); be a multi-core processor; or include one or more processors 1102. Although this disclosure describes and illustrates a particular processor, this disclosure contemplates any suitable processor.

In particular embodiments, memory 1104 includes main memory for storing instructions for processor 1102 to execute or data for processor 1102 to operate on. As an example and not by way of limitation, computer system 1100 may load instructions from storage 1106 or another source (such as, for example, another computer system 1100) to memory 1104. Processor 1102 may then load the instructions from memory 1104 to an internal register or internal cache. To execute the instructions, processor 1102 may retrieve the instructions from the internal register or internal cache and decode them. During or after execution of the instructions, processor 1102 may write one or more results (which may be intermediate or final results) to the internal register or internal cache. Processor 1102 may then write one or more of those results to memory 1104. In particular embodiments, processor 1102 executes only instructions in one or more internal registers or internal caches or in memory 1104 (as opposed to storage 1106 or elsewhere) and operates only on data in one or more internal registers or internal caches or in memory 1104 (as opposed to storage 1106 or elsewhere). One or more memory buses (which may each include an address bus and a data bus) may couple processor 1102 to memory 1104. Bus 1112 may include one or more memory buses, as described below. In particular embodiments, one or more memory management units (MMUs) reside between processor 1102 and memory 1104 and facilitate accesses to memory 1104 requested by processor 1102. In particular embodiments, memory 1104 includes random access memory (RAM). This RAM may be volatile memory, where appropriate Where appropriate, this RAM may be dynamic RAM (DRAM) or static RAM (SRAM). Moreover, where appropriate, this RAM may be single-ported or multi-ported RAM. This disclosure contemplates any suitable RAM. Memory 1104 may include one or more memories 1104, where appropriate. Although this disclosure describes and illustrates particular memory, this disclosure contemplates any suitable memory.

In particular embodiments, storage 1106 includes mass storage for data or instructions. As an example and not by way of limitation, storage 1106 may include a hard disk drive (HDD), a floppy disk drive, flash memory, an optical disc, a magneto-optical disc, magnetic tape, or a Universal Serial Bus (USB) drive or a combination of two or more of these. Storage 1106 may include removable or non-removable (or fixed) media, where appropriate. Storage 1106 may be internal or external to computer system 1100, where appropriate. In particular embodiments, storage 1106 is non-volatile, solid-state memory. In particular embodiments, storage 1106 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 1106 taking any suitable physical form. Storage 1106 may include one or more storage control units facilitating communication between processor 1102 and storage 1106, where appropriate. Where appropriate, storage 1106 may include one or more storages 1106. Although this disclosure describes and illustrates particular storage, this disclosure contemplates any suitable storage.

In particular embodiments, I/O interface 1108 includes hardware, software, or both, providing one or more interfaces for communication between computer system 1100 and one or more I/O devices. Computer system 1100 may include one or more of these I/O devices, where appropriate. One or more of these I/O devices may enable communication between a person and computer system 1100. As an example and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 1108 for them. Where appropriate, I/O interface 1108 may include one or more device or software drivers enabling processor 1102 to drive one or more of these I/O devices. I/O interface 1108 may include one or more I/O interfaces 1108, where appropriate. Although this disclosure describes and illustrates a particular I/O interface, this disclosure contemplates any suitable I/O interface.

In particular embodiments, communication interface 1110 includes hardware, software, or both providing one or more interfaces for communication (such as, for example, packet-based communication) between computer system 1100 and one or more other computer systems 1100 or one or more networks. As an example and not by way of limitation, communication interface 1110 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 1110 for it. As an example and not by way of limitation, computer system 1100 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 1100 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 1100 may include any suitable communication interface 1110 for any of these networks, where appropriate. Communication interface 1110 may include one or more communication interfaces 1110, where appropriate. Although this disclosure describes and illustrates a particular communication interface, this disclosure contemplates any suitable communication interface.

In particular embodiments, bus 1112 includes hardware, software, or both coupling components of computer system 1100 to each other. As an example and not by way of limitation, bus 1112 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 1112 may include one or more buses 1112, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate.

Herein, “or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

The scope of this disclosure encompasses all changes, substitutions, variations, alterations, and modifications to the example embodiments described or illustrated herein that a person having ordinary skill in the art would comprehend. The scope of this disclosure is not limited to the example embodiments described or illustrated herein. Moreover, although this disclosure describes and illustrates respective embodiments herein as including particular components, elements, feature, functions, operations, or steps, any of these embodiments may include any combination or permutation of any of the components, elements, features, functions, operations, or steps described or illustrated anywhere herein that a person having ordinary skill in the art would comprehend.

What is claimed is:

1. A method comprising:
detecting, by a motion sensor of a mobile device worn by a user, a plurality of motion signals, each representing a motion of the user about one of a plurality of mobile-device axes defined by an orientation of the mobile device;
determining, for each of the plurality of mobile-device axes, a ballistocardiogram (BCG) signal based on the motion signal corresponding to that mobile-device axis;
selecting, based on a strength of the determined BCG signals, one or more particular mobile-device axes and corresponding motion signals for estimating a user's tidal volume;
determining, based on the one or more selected motion signals, one or more breathing features; and
estimating, by providing the one or more breathing features to a trained machine-learning model, the user's current tidal volume.

2. The method of claim 1, wherein the mobile device comprises a head-worn device.

3. The method of claim 2, wherein the head-worn device comprises one or more earbuds.

4. The method of claim 3, wherein the plurality of mobile-device axes comprises a set of three axes in Cartesian coordinates.

5. The method of claim 4, wherein determining, for each of the plurality of mobile-device axes, a ballistocardiogram (BCG) signal based on the motion signal corresponding to that mobile-device axis comprises detecting, for each of the plurality of mobile-device axes, a plurality of J-peaks in each motion signals.

6. The method of claim 4, wherein the motion signals correspond to a segment of static motion signals.

7. The method of claim 6, further comprising determining the segment of static motion signals by:
dividing an output of the motion sensor into a plurality of intervals;
labeling each interval as either static or not static;
determining a longest section of the output of the motion sensor comprising a continuous sequence of static labels;
selecting the longest section as the segment of static motion signals.

8. The method of claim 7, wherein determining a longest section of the output of the motion sensor comprising a continuous sequence of static labels further comprises determining that the longest section of the output of the motion sensor comprising a continuous sequence of static labels exceeds a predetermined threshold period of time.

9. The method of claim 8, wherein the predetermined threshold period of time comprises at least 10 seconds.

10. The method of claim 1, wherein selecting, based on a strength of the determined BCG signals, one or more particular mobile-device axes and corresponding motion signals for estimating the user's tidal volume comprises selecting the one mobile-device axis and corresponding motion signal that corresponds to the strongest BCG signal.

11. The method of claim 1, wherein selecting, based on a strength of the determined BCG signals, one or more particular mobile-device axes and corresponding motion signals for estimating the user's tidal volume comprises selecting the one mobile-device axis based on a difference between an ensemble-based strongest BCG signal and an average-based strongest BCG signal.

12. The method of claim 1, wherein selecting, based on a strength of the determined BCG signals, one or more particular mobile-device axes and corresponding motion signals for estimating the user's tidal volume comprises selecting a strongest BCG signal J-peak amplitude above a particular threshold.

13. The method of claim 1, wherein selecting, based on a strength of the determined BCG signals, one or more particular mobile-device axes and corresponding motion signals for estimating the user's tidal volume comprises selecting the two mobile-device axes and corresponding motion signals that corresponds to the two strongest BCG signals.

14. The method of claim 1, wherein selecting, based on a strength of the determined BCG signals, one or more particular mobile-device axes and corresponding motion signals for estimating a user's tidal volume, comprises:
determining, based on a relative strength of the determined BCG signals, a transformation matrix for the mobile sensor; and
transforming the orientation of the mobile device by the transformation matrix to select the one or more particular mobile-device axes and corresponding motion signals.

15. The method of claim 1, wherein the mobile device comprises a mobile phone or a watch placed on the user's chest.

16. The method of claim 1, further comprising:
recording audio of the user's breathing;
synchronizing the recorded audio with the plurality of motion signals; and
determining, based on the one or more selected motion signals and the audio of the user's breathing, one or more breathing feature.

17. The method of claim 1, further comprising:
determining, based on the plurality of motion signals, at least one of (1) one or more time-domain features and (2) one or more frequency-domain features; and
estimating the user's current tidal volume estimating by providing the one or more breathing features and one ore more determined time-domain features, if any, and one or more frequency-domain features, if any, to a trained machine-learning model.

18. An apparatus comprising: one or more non-transitory computer readable storage media storing instructions; and one or more processors coupled to the one or more non-transitory computer readable storage media and operable to execute the instructions to:
access a plurality of motion signals detected by a motion sensor of a mobile device worn by a user, each representing a motion of the user about one of a plurality of mobile-device axes defined by an orientation of the mobile device;
determine, for each of the plurality of mobile-device axes, a ballistocardiogram (BCG) signal based on the motion signal corresponding to that mobile-device axis;
select, based on a strength of the determined BCG signals, one or more particular mobile-device axes and corresponding motion signals for estimating a user's tidal volume;
determine, based on the one or more selected motion signals, one or more breathing features; and
estimate, by providing the one or more breathing features to a trained machine-learning model, the user's current tidal volume.

19. One or more non-transitory computer readable storage media storing instructions that are operable when executed to:
access a plurality of motion signals detected by a motion sensor of a mobile device worn by a user, each representing a motion of the user about one of a plurality of mobile-device axes defined by an orientation of the mobile device;
determine, for each of the plurality of mobile-device axes, a ballistocardiogram (BCG) signal based on the motion signal corresponding to that mobile-device axis;
select, based on a strength of the determined BCG signals, one or more particular mobile-device axes and corresponding motion signals for estimating a user's tidal volume;
determine, based on the one or more selected motion signals, one or more breathing features; and
estimate, by providing the one or more breathing features to a trained machine-learning model, the user's current tidal volume.

20. The media of claim 19, wherein the mobile device comprises one or more earbuds.
